(19) United States
(12) Patent Application Publication
(10) Pub. No.: US 2003/0129236 A1
(43) Pub. Date: Jul. 10, 2003

(54) Multiple pulse extended release formulations of clindamycin

(76) Inventors: John M. Heimlich, Portage, MI (US); Robert M. Noack, Grand Rapids, MI (US); Steven R. Cox, Schoolcraft, MI (US); Loksidh D. Ganorkar, Kalamazoo, MI (US); Ronald R. VerHage, Lawton, MI (US); Ernest J. Lee, Kalamazoo, MI (US)

Correspondence Address: PHARMACIA CORPORATION, GLOBAL PATENT DEPARTMENT, POST OFFICE BOX 1027, ST. LOUIS, MO, 63006, US

(21) Appl. No.: 10/324,718

(22) Filed: Dec. 19, 2002

Related U.S. Application Data
(60) Provisional application No. 60/342,642, filed on Dec. 20, 2001.

(51) Int. Cl. A61K 31/704; A61K 9/22; A61K 9/26

(52) U.S. Cl. 424/470; 514/35

(57) ABSTRACT

The present invention is directed to an oral dosage form for multiple-pulsed delivery of at least two fractions of clindamycin to a subject, one in an immediate-release form and the other in an extended release form. The oral dosage forms of the present invention provide a means for treating or preventing gram-positive bacterial infections with a minimal number of treatments per day, potentially, as little as once or twice per day.

This application claims the benefit of U.S. Provisional Application Serial No. 06/342,642, filed Dec. 20, 2001.

### FIELD OF THE INVENTION

The present invention relates to orally deliverable pharmaceutical compositions of antibiotics, such as clindamycin, to processes for preparing such compositions, and to methods of treatment of disorders caused by bacteria by orally administering such compositions to a subject. In one aspect, the present invention relates to orally deliverable pharmaceutical compositions designed to release clindamycin in the colon of a subject.

### BACKGROUND OF THE INVENTION

Clindamycin has long been recognized as a broad spectrum antibiotic, useful for the treatment of a variety of disorders related to bacterial infections. Oral immediate release formulations of clindamycin have been developed, designed to release clindamycin in a single pulse into the upper gastrointestinal (“GI”) tract. (See, e.g. commercial oral formulations of clindamycin hydrochloride designed for adults, such as CLEOCIN® HCl from Pharmacia Corporation; and oral formulations of clindamycin palmitate hydrochloride designed for children, such as CLEOCIN® PEDIATRIC). Both clindamycin hydrochloride and clindamycin palmitate hydrochloride are hydrolyzed to clindamycin free base in the gastrointestinal tract of a subject, prior to being absorbed into the bloodstream.

Clindamycin is very soluble in aqueous media. Unformulated clindamycin is quickly dissolved and dispersed for rapid absorption in the gastrointestinal tract when administered orally. Commercial formulations of clindamycin, such as the two described above, are designed for immediate release of the drug into the gastrointestinal tract. Immediate release formulations are highly effective in administering an active agent to a subject, quickly elevating the levels of the active agent in the intestinal tract and plasma of the subject. However, the dosage of an active agent such as clindamycin in the intestinal tract and plasma of a subject drops off over time, such that another dose is required six to eight hours later in order to maintain an effective amount of clindamycin in the subject.

Extended release oral formulations of active agents other than clindamycin are known, including dual release dosage forms. As used herein, the term “dual release dosage form” refers to a dosage form which releases an active ingredient contained therein, such as clindamycin, in two different boluses or pulses, separated in time from one another. The dual release dosage form enables one to deliver a drug or other active agent to a subject in two pulses, rather than one, eliminating the necessity for administration of a second dose of the drug, if the doses are properly separated.

Examples of known dual release formulations follow. U.S. Pat. No. 6,228,398 by Devane et al. discloses a microparticulate modified release composition that releases active ingredients at two different times, after oral administration. U.S. Pat. No. 4,871,549 by Ueda et al. discloses a time-controlled explosion system for pharmaceutical compositions that can be used in either zero-order or dual release administration of an active agent. WO 00/25757, filed by MERCK & CO., INC. discloses a composition comprising a swellable polymer in a core with an active agent, covered by water insoluble film with a plurality of apertures. The first pulse of active agent is released from this last composition by passive diffusion through the apertures, while the second pulse is released when the core polymer swells and is extruded through the plurality of apertures. (WO 00/25757, Abstract).

Various means for targeted release of active agents into the colon of a subject have been disclosed. See, e.g. EP 0572942 B1, filed by POLI INDUSTRIA CHIMICA S.p.A.; U.S. Pat. No. 5,900,252, by Calanchi et al.; WO 98/22095, filed by THE PROCTOR & GAMBLE COMPANY; WO 98/26767, filed by POLI INDUSTRIA CHIMICA S.p.A; U.S. Pat. No. 5,505,966, by Edman et al.; and WO 97/25979, filed by PERIO PRODUCTS LTD. However, in each such case, release was in the form of a single bolus (i.e., single pulse delivery) of active agent delivered to the intended target. The colon is typically not an attractive target for release of many different drugs (unless local action is desired), because of the fact that many drugs are more poorly absorbed into the bloodstream in the colon than in other parts of the gastrointestinal tract. Also, until now, it has been thought that clindamycin should not be released directly into the colon because of the risk of causing Pseudomembranous Colitis.

A need exists for a multiple pulse oral formulations of clindamycin; particularly for such a formulation that enables one to maintain a therapeutically or prophylactically effective amount of clindamycin in the bloodstream of a subject for an extended period of time after oral administration thereto. Specifically, a need exists for such a multiple pulse oral formulation, wherein at least one portion of clindamycin is released in the colon of a subject, thereby facilitating once daily dosing of clindamycin. Once daily dosing, or at least less frequent dosing than currently available formulations of clindamycin are expected to increase patient convenience and compliance, as well as potentially improving efficacy and decreasing side effects.

### SUMMARY OF THE INVENTION

The present invention comprises a composition designed to release clindamycin in multiple pulses, over an extended period of time after oral administration to a subject. The composition of the present invention is specifically designed to release a first fraction of the clindamycin in a first pulse, within about 30 minutes of oral administration, and a second fraction of clindamycin in a second pulse, about 4 hours to 15 hours after oral administration. Release of additional pulses of clindamycin into the subject is also possible using compositions of the present invention.

Clindamycin is one of the most useful, most broad spectrum antibiotics known. It has found utility in use in treatment of a wide variety of bacterial infections and various disorders associated therewith. The composition of the present invention provides an orally deliverable means for extended release of clindamycin into a subject. The compositions of the present invention, thus, can potentially eliminate the necessity for intake of multiple doses of the same drug, increasing the chance of patient compliance with a dosing regimen.

### BRIEF DESCRIPTION OF THE DRAWINGS

FIG. 1 is a diagram of the lower part of the gastrointestinal tract of a human being, showing regions where clindamycin HCl was released into human subjects from Enterion capsules, as described in the Examples section, below.

### DETAILED DESCRIPTION OF THE INVENTION

The term “oral administration” herein includes any form of delivery of a therapeutic agent or a composition thereof to a subject wherein the agent or composition is swallowed by a subject, regardless of whether the composition is placed in the mouth prior to swallowing. Thus “oral administration” includes esophageal administration. Absorption of the agent can occur in any part or parts of the gastrointestinal tract including the mouth, esophagus, stomach, duodenum, ileum and colon.

The term “orally deliverable” herein means suitable for oral administration.

A “subject” herein to which a therapeutic agent or composition thereof can be administered includes a human patient of either sex and of any age, and also includes any nonhuman animal, particularly a domestic or companion animal, illustratively a cat, dog or horse.

The term “dose unit” herein means a portion of a pharmaceutical composition that contains an amount of a therapeutic agent, in the present case a form of clindamycin, suitable for a single oral administration to provide a therapeutic effect. Typically one dose unit, or a small plurality (up to about 4) of dose units, provides a sufficient amount of the agent to result in the desired effect.

The term “present in solid particles” as applied to a drug herein encompasses compositions wherein the solid particles consist essentially of the drug and compositions wherein the solid particles comprise the drug in intimate mixture with one or more other ingredients. These other ingredients can include one or more therapeutic agents other than the drug and/or one or more pharmaceutically acceptable excipients.

The term “excipient” herein means any substance, not itself a therapeutic agent, used as a carrier or vehicle for delivery of a therapeutic agent to a subject or added to a pharmaceutical composition to improve its handling, storage, disintegration, dispersion, dissolution, release or organoleptic properties or to permit or facilitate formation of a dose unit of the composition into a discrete article such as a capsule or tablet suitable for oral administration. Excipients can include, by way of illustration and not limitation, diluents, disintegrants, binding agents, adhesives, wetting agents, polymers, lubricants, glidants, substances added to mask or counteract a disagreeable taste or odor, flavors, dyes, fragrances, and substances added to improve appearance of the composition.

The term “substantially homogeneous” with reference to a pharmaceutical composition that comprises several components means that the components are sufficiently mixed such that individual components are not present as discrete layers and do not form concentration gradients within the composition.

The multiple-pulse release composition of the present invention enables the release of the first fraction of clindamycin in a first pulse at the first time point, and the second fraction of clindamycin in a second pulse at the second time point after administration to a subject, as described above. In a preferred embodiment at least one additional fraction of clindamycin is released in at least one additional delayed-release pulse, after oral administration to a subject.

Compositions of this embodiment of the invention are referred to, for convenience herein as “multiple-pulse release compositions”. Multiple-pulse release compositions designed to release clindamycin in only a first and second fraction are referred to herein as “dual-release compositions.” When formulated as tablets, which are a preferred dosage form for this embodiment, such compositions are referred to herein as “multiple-pulse release tablets” or as “dual-pulse release tablets”, respectively.

Compositions of the present invention comprise at least one orally deliverable dose unit of clindamycin. Each dose unit comprises a discrete amount of clindamycin, preferably a pharmaceutically effective amount. Clindamycin provided in the compositions of the formulations is in any one of a number of bioavailable forms, including but not limited to clindamycin HCl, clindamycin phosphate, clindamycin palmitate, clindamycin free base (amorphous), and clindamycin crystalline free base. The clindamycin is preferably present in at least one form as clindamycin HCl, clindamycin phosphate, or clindamycin crystalline free base, more preferably as clindamycin HCl or as clindamycin crystalline free base, even more preferably as clindamycin HCl.

Crystalline clindamycin free base is disclosed in U.S. patent application Ser. No. 10/228,356, incorporated herein by reference. Crystalline clindamycin free base can be produced by either of the two alternative processes, illustrated in the above-referenced patent application. One illustrative process of preparing crystalline clindamycin free base involves forming the amorphous free base as a precipitate in aqueous medium followed by agitation to crystallize the free base from the precipitate. An illustrative example of the method involves first dissolving a salt of clindamycin, e.g., clindamycin hydrochloride in a solvent, preferably a polar solvent such as, for example, water. This if followed by adding an alkali material, i.e. a base, in an aqueous vehicle such as for example, a NaOH solution, such as, for example, preferably from about 0.01 to about 10 N NaOH solution, more preferably from about 0.1 to about 1 N NaOH, and more preferably about 0.5 N NaOH. This results in precipitation of the amorphous free base. The amorphous free base is then crystallized by agitation of the precipitate by, for example, by sonicating or manually shaking the precipitate, or by both sonicating and manually shaking the precipitate suspended in the aqueous medium. The crystallized free base is then preferably harvested by centrifugation, followed by removal of the liquid portion. The crystallized free base is preferably washed in at least one washing step involving adding a wash solution, sonicating, shaking, centrifuging and removing the wash solution from the crystalline material. The wash solution is preferably aqueous, more preferably water.

In an alternate method, crystalline clindamycin free base can be produced by a slow addition of a clindamycin salt, such as clindamycin hydrochloride, dissolved in a polar solvent such as water to an aqueous alkaline solution containing a water-soluble organic substance, preferably an alcohol co-solvent. The aqueous solution containing an alkali with an alcohol co-solvent is prepared by adding the alkali, i.e. base, in an aqueous vehicle such as, for example, a NaOH solution. The NaOH solution can be, for example, preferably from about 0.01 to about 10 N NaOH solution, more preferably from about 0.1 to about 1 N NaOH, and more preferably about 0.5 N NaOH. The alcohol co-solvent is present, preferably in an amount of from about 2% to about 20%, more preferably from about 5% to about 10%. Any of a number of alcohols that are readily miscible with water can be used, preferably, methanol, ethanol, n-propanol, t-butanol and the like. Typically alcohols of higher molecular weight are less soluble in water and less preferred. Diols such as 1,2, ethanediol (ethylene glycol), 1,2 propanediol (propylene glycol) and 1,2 butanediol and triols such as 1,2,3 propantriol (glycerol) and the like can also be used as co-solvent. It is also possible to use an aqueous solution of a water-soluble organic substance such as, for example, sodium acetate.

An aqueous solution of a clindamycin salt, such as, for example clindamycin hydrochloride is prepared and slowly added to the alkali solution with alcohol co-solvent, preferably over a period of from about 15 minutes to about 4 hours, more preferably from about 30 minutes to about 2 hours and most preferably from about 45 minutes to 75 minutes. Crystallization is allowed to proceed for 1 to 24 hours and the crystalline free base material is isolated by filtration, centrifugation and decanting or the like. In a preferred variation of this method, the clindamycin hydrochloride solution is added in a multi-phase infusion schedule such as, for example, a first phase of slow infusion over about one hour, followed by a faster infusion phase over about 30 min and concluding with slow infusion phase over about one hour.

The material obtained by either of the methods above is isolated and dried, for example, under a stream of humidified nitrogen. The dry material can be further processed such as by grinding to produce a dry powder.

A single form or two or more forms of clindamycin can be used in the compositions of the present invention. Selection of the form or combination of forms of clindamycin to use in any given composition of the present invention depends, at least in part, upon the desired release properties and the solubility of each form of clindamycin. Clindamycin HCl is highly soluble in water, while clindamycin crystalline free base is considerably less soluble. Amorphous clindamycin free base is the least soluble of all the forms of clindamycin listed above. By using two or more different forms of clindamycin in a composition of the present invention, each of which has a different solubility in water, one can vary the release rate of clindamycin after oral administration. However, release rates can also be controlled using various excipients, polymers, and matrices, such as are described below. Thus, it is contemplated but not necessary for the formulations of the present invention to comprise more than one form of clindamycin.

Compositions of the present invention comprise one or more orally deliverable dose units, and enable the delivery of at least two dose units of clindamycin. The compositions can deliver the same or different amounts of clindamycin in each dose unit. The first dose unit is delivered in a first pulse, and the second dose unit is delivered in a second pulse. Each dose unit delivered by the composition is preferably a therapeutically effective amount.

As used herein, the term “first time” and “second time” refer to time periods after oral administration of a composition of the present invention to a subject, wherein clindamycin is released. The duration such release at any given time can be long or short. When the duration of release at either the first time or the second time is short, the result is a pulsed release of a single dose unit of clindamycin at that time.

It will be understood that a therapeutically effective amount of clindamycin composition for a subject is dependent inter alia on the body weight of the subject. Where the subject is a child or a small animal (e.g., a dog), for example, the amount of clindamycin required to provide blood serum concentrations consistent with therapeutic effectiveness is relatively less than the amount required to provide comparable blood serum concentrations in an adult human or a large animal. The compositions of the present invention comprise a first fraction of clindamycin in a form released in a first pulse, preferably in an immediate-release form, and a second fraction of clindamycin in a form released in a second pulse, after oral administration of the composition to a subject.

Release of the first pulse of the formulation of the present invention preferably occurs within about 30 minutes of oral administration, more preferably within about 10 minutes of oral administration. Release of the second fraction preferably occurs about 4 hours to about 15 hours, more preferably about 6 hours to about 12 hours, even more preferably about 8 hours to about 12 hours after oral administration. The second fraction is preferably released after the composition passes through the stomach of a subject, more preferably after the composition has entered the small intestines, and even more preferably after the composition has entered the colon.

The rate of movement of the composition through the gastrointestinal tract of any given subject will vary, such that in order to deliver the second pulse of clindamycin to a particular target, such as the colon, it is contemplated that the second pulse will need to be released at a time point later than the preferred ranges given above. In order to ensure colonic release in a human adult subject, it is most preferred that at least one pulse of clindamycin be released at least about 12 hours after oral administration.

Dose units can be selected to accommodate any desired frequency of administration used to achieve a desired daily dosage. The daily dosage and frequency of administration, and therefore the selection of an appropriate dose unit, depends on a variety of factors, including the age, weight, sex and medical condition of the subject, and the nature and severity of the condition or disorder, and thus may vary widely.

The composition preferably contains about 1% to about 95%, preferably about 10% to about 90%, more preferably about 25% to about 85%, and still more preferably about 30% to about 80%, by weight of clindamycin. A composition of the invention is preferably made in the form of discrete dose units each containing a predetermined amount of clindamycin, such as tablets, pills, hard or soft capsules, cachets, dispensable powders, granules, or suspensions or any other form reasonably adapted for oral administration and extended release. Tablets, pills and the like additionally can be prepared with or without coatings.

One embodiment of the multiple pulse release technology is a tablet in a capsule, where the tablet is manufactured via traditional manufacturing technology, the tablet is then coated with a functional coating and the pulse release is provided via an effervescent or swelling rupture of the coating or via the coating being solubilized at a certain pH through the use of a pH dependent coating. The swelling rupture is caused via swellable excipients that are incorporated into the formulation while the effervescent rupture is caused by the incorporation of effervescent agents into the tablet formulation.

Other embodiments are based on multiparticulate technologies; compressed pellets, extruded beads, or spray dried beads (drug particles). Each of these technologies are coated and rely on effervescent or swelling rupture of the coating or a coating that is solubilized at a certain pH via the use of a pH dependent coating. The swelling rupture is caused via swellable excipients that are incorporated into the formulation while the effervescent rupture is caused by the incorporation of effervescent agents into the tablet formulation.

In an alternative embodiment, the second fraction is in a sustained release form, such that delivery of the fraction of clindamycin in the second pulse is spread out over time, after administration to a subject. In this embodiment, any additional fractions are preferably also delivered in a sustained release form. The above-mentioned technologies are suitably used with the incorporation of release limiting ingredients to provide for a sustained release second fraction.

In another particularly preferred embodiment of the present invention, the second fraction of clindamycin is present in a multiplicity of solid beads, pellets or granules each having a coating comprising a polymer, preferably a release-extending polymer. Such beads, pellets or granules are referred to herein as “beads” or “coated beads” and are typically dense, hard, substantially spherical and of low friability. Compositions of this embodiment of the invention are referred to for convenience herein as “dual-pulse release coated bead compositions”. When formulated as capsules, which are a preferred dosage form for this embodiment, such compositions are referred to herein as “dual-pulse release coated bead capsules”. Such dual-pulse release beads are described in greater detail below.

In the multiple-pulse release coated bead composition of the present invention, whether encapsulated or tableted, the first fraction of clindamycin can be present in any suitable immediate-release form. The demands of a dual-pulse release clindamycin drug composition are met surprisingly well by a dual-pulse release coated bead preparation wherein the beads containing the sustained-release fraction of clindamycin drug are coated with a barrier layer comprising at least one release extending polymer. The beads optionally contain pharmaceutically acceptable excipients such as lactose and microcrystalline cellulose. The beads can be prepared by mixing and granulation of clindamycin with one or more excipients, followed by extrusion, spheronization, drying and sieving the particles to the desired size range, followed by application of a polymer, preferably a release-extending polymer coating to the beads containing that fraction of clindamycin that is desired to exhibit sustained release.

In another embodiment, the beads have a core comprising a pharmaceutically acceptable excipient such as starch or sucrose, surrounded by one or more shells each comprising an inner drug-containing layer and an outer polymer barrier layer, preferably a release-extending polymer barrier layer. In a sustained-release coating preferred in the dual pulse release compositions of the present invention, the beads containing the second fraction of clindamycin together with one or more excipients are coated with one or more polymers selected from hydroxypropylcellulose, hydroxyethylcellulose, methylhydroxyethylcellulose, methylcellulose, ethylcellulose (e.g., Surelease™ of Colorcon), cellulose acetate, sodium carboxymethylcellulose, polymers and copolymers of acrylic acid and methacrylic acid and esters thereof (e.g., Eudragit™ RL, Eudragit™ RS, Eudragit™ L100, Eudragit™ S100, Eudragit™ NE), polyvinylpyrrolidone and polyethylene glycols. The polymers can be combined with water-soluble substances such as hydroxypropylmethylcellulose (hereinafter, “HPMC”), polyethylene glycol, etc. to form pores or channels in the coating to modify the release rate.

Eudragit™ of Rohm Pharma is a trade name applied to a range of products useful for film coating of sustained-release particles. These products are of varying solubility in gastrointestinal fluids. Eudragit™ RL and Eudragit™ RS are copolymers synthesized from acrylic and methacrylic esters with a low content of quaternary ammonium groups. Eudragit™ RL and Eudragit™ RS differ in the mole ratios of such ammonium groups to the remaining neutral (meth)acrylic acid esters (1:20 and 1:40 respectively). Eudragit™ NE is an aqueous dispersion of a neutral copolymer based on ethyl acrylate and methyl methacrylate. Characteristics of Eudragit™ polymers are described in Eudragit: Sustained-release Formulations for Oral Dosage Forms, Rohm Basic Info 2.

Ethylcellulose, available as an aqueous dispersion, for example under the trade name Surelease™, is another suitable material which is available in different grades and in special qualities for preparing barrier coatings. According to the invention it is preferred to use ethylcellulose having a viscosity of about 5 cP to about 15 cP, but other types of cellulose-based polymers can be used. It is especially preferred to use ethylcellulose in combination with HPMC.

The coating procedure can be performed by conventional means employing, for example, spraying equipment, a fluidized bed and equipment for drying and size fractionating. The liquid used in the coating procedure contains one or more barrier layer forming components and one or more solvents, such as ethanol, acetone, methyl isobutyl ketone (MIBK), water and others well known in this technical field. The coating liquid can be in the form of a solution, a dispersion, an emulsion or a melt, depending on the specific nature of the coating constituents.

Plasticizers and pigments can optionally be used to modify the technical properties or change the permeability of the coating.

Each coated bead containing clindamycin represents an individual controlled release unit, releasing the drug at a predetermined rate, preferably independent of its position in the gastrointestinal tract. Overall dissolution profile and drug availability are dependent on the rate of drug diffusion through the sustained-release coating and/or on the rate of erosion of the coating in the gastrointestinal tract and/or on the controlled rupture of the coating.

The coated beads manufactured as above, together with immediate-release beads, are encapsulated by a conventional encapsulation process.

In an illustrative process for preparing a composition of the invention having dual-release beads, the first fraction of drug is dispersed in a liquid medium in which the drug is substantially insoluble, to form a first drug suspension, which is then wet milled. Milling conditions can be readily optimized by one skilled in the art to provide drug particles of a desired size range. The wet milled drug suspension is then spray coated onto sugar spheres. Next, a liquid polymer coating comprising one or more release-extending polymers and water is prepared. The polymer coating is then sprayed on top of the drug-coated sugar beads using any suitable spraying apparatus to form sustained-release beads.

Next, a second drug suspension comprising the second fraction of the drug is prepared in similar fashion to the first drug suspension. Additionally, a disintegrant suspension, for example comprising a disintegrant (e.g., croscarmellose sodium) and water, is prepared and wet milled. The second drug suspension and the milled disintegrant suspension are then mixed together to form a drug/disintegrant suspension. The drug/disintegrant suspension is then sprayed on top of the sustained-release beads prepared as above using any suitable spray coating equipment. All spray coating conditions can be readily optimized by one skilled in the art to provide a desired rate of coating and coat thickness.

The multiple-release beads manufactured as above are encapsulated by a conventional encapsulation process.

Excipients useful in compositions of the invention can be liquids, semi-solids, solids or combinations thereof. Excipient-containing compositions of the invention can be prepared by any suitable method of pharmacy which includes the step of bringing into association one or more excipients with clindamycin, in a combination of dissolved, suspended, nanoparticulate, microparticulate or controlled-release, slow-release, programmed-release, timed-release, pulse-release, sustained-release or extended-release forms thereof. In general, such compositions are prepared by uniformly and intimately admixing the drug with a liquid or finely divided diluent, or both, and then, if necessary or desired, encapsulating or shaping the product. For example, a tablet can be prepared by compressing or molding a powder or granules of clindamycin, together with one or more excipients. Compressed tablets can be prepared by compressing, in a suitable machine, a free-flowing composition, such as a powder or granules, comprising the drug optionally mixed with one or more binding agent(s), lubricant(s), inert diluent(s), wetting agent(s) and/or dispersing agent(s). Molded tablets can be made by molding, in a suitable machine, the drug moistened with an inert liquid diluent.

Compositions of the invention typically comprise clindamycin in a desired amount admixed with one or more excipients selected from the group consisting of pharmaceutically acceptable diluents, disintegrants, binding agents, adhesives, wetting agents, lubricants, and anti-adherent agents. In addition, nanoparticles, microparticles and/or controlled-release, slow-release, programmed-release, timed-release, pulse-release, sustained-release or extended-release particles of the drug, if present, can optionally contain one or more matrix polymers and/or surface modifying agents. Drug particles can be aggregated into beads which are enveloped in a coating conferring controlled-release, slow-release, programmed-release, timed-release, pulse-release, sustained-release or extended-release properties to the drug in such beads.

Through selection and combination of excipients, compositions can be provided exhibiting improved performance with respect to, among other properties, efficacy, bioavailability, clearance time, stability, compatibility of drug and excipients, safety, dissolution profile, disintegration profile and/or other pharmacokinetic, chemical and/or physical properties. Where the composition is formulated as a tablet, the combination of excipients selected provides tablets that can exhibit improvement, among other properties, in dissolution profile, hardness, crushing strength, and/or friability.

Compositions of the invention optionally comprise one or more pharmaceutically acceptable diluents as excipients. Suitable diluents illustratively include, either individually or in combination, lactose, including anhydrous lactose and lactose monohydrate; starches, including directly compressible starch and hydrolyzed starches (e.g., Celutab™ and Emdex™); mannitol; sorbitol; xylitol; dextrose (e.g., Cerelose™ 2000) and dextrose monohydrate; dibasic calcium phosphate dihydrate; sucrose-based diluents; confectioner's sugar; monobasic calcium sulfate monohydrate; calcium sulfate dihydrate; granular calcium lactate trihydrate; dextrates; inositol; hydrolyzed cereal solids; amylose; celluloses including microcrystalline cellulose, and amorphous cellulose (e.g., Rexcel™) and powdered cellulose; calcium carbonate; glycine; bentonite; and polyvinylpyrrolidone. Such diluents, if present, constitute in total about 5% to about 99%, preferably about 10% to about 85%, and more preferably about 20% to about 80%, of the total weight of the composition. The diluent or diluents selected preferably exhibit suitable flow properties and, where tablets are desired, compressibility.

Microcrystalline cellulose is a preferred diluent. This diluent is chemically compatible with clindamycin. The use of extragranular microcrystalline cellulose (that is, microcrystalline cellulose added to a granulated composition) can be used to improve hardness (for tablets) and/or disintegration time. It typically provides compositions having suitable release rates of clindamycin, stability, flowability, and/or drying properties at a relatively low diluent cost. It provides a high density substrate that aids densification during granulation and therefore improves blend flow properties.

Compositions of the invention optionally comprise one or more pharmaceutically acceptable disintegrants as excipients, particularly for tablet formulations. Suitable disintegrants include, either individually or in combination, starches, including sodium starch glycolate (e.g., Explotab™ of PenWest) and pregelatinized corn starches (e.g., National™ 1551, National™ 1550, and Colorcon™ 1500), clays (e.g., Veegum™ HV), celluloses such as purified cellulose, microcrystalline cellulose, methylcellulose, carboxymethylcellulose and sodium carboxymethylcellulose, croscarmellose sodium (e.g., Ac-Di-Sol™ of FMC), alginates, crospovidone, and gums such as agar, guar, locust bean, karaya, pectin and tragacanth gums.

Disintegrants may be added at any suitable step during the preparation of the. composition, particularly prior to granulation or during a lubrication step prior to compression. Such disintegrants, if present, constitute in total about 0.2% to about 30%, preferably about 0.2% to about 10%, and more preferably about 0.2% to about 5%, of the total weight of the composition.

Croscarmellose sodium is a preferred disintegrant for tablet or capsule disintegration, and, if present, preferably constitutes about 0.2% to about 10%, more preferably about 0.2% to about 7%, and still more preferably about 0.2% to about 5%, of the total weight of the composition. Croscarmellose sodium confers superior intragranular disintegration capabilities to granulated compositions of the present invention.

Compositions of the invention optionally comprise one or more pharmaceutically acceptable binding agents or adhesives as excipients, particularly for tablet formulations. Such binding agents and adhesives preferably impart sufficient cohesion to the powder being tableted to allow for normal processing operations such as sizing, lubrication, compression and packaging, but still allow the tablet to disintegrate and the composition to be absorbed upon ingestion. Suitable binding agents and adhesives include, either individually or in combination, acacia; tragacanth; sucrose; gelatin; glucose; starches such as, but not limited to, pregelatinized starches (e.g., National™ 1511 and National™ 1500); celluloses such as, but not limited to, microcrystalline cellulose, methylcellulose and carmellose sodium (e.g., Tylose™); alginic acid and salts of alginic acid; magnesium aluminum silicate; PEG; guar gum; polysaccharide acids; bentonites; povidone, for example povidone K-15, K-30 and K-29/32; polymethacrylates; HPMC; hydroxypropylcellulose (e.g., Klucel™); and ethylcellulose (e.g., Ethocel™). Such binding agents and/or adhesives, if present, constitute in total about 0.5% to about 25%, preferably about 0.75% to about 15%, and more preferably about 1% to about 10%, of the total weight of the composition.

Compositions of the invention optionally comprise one or more pharmaceutically acceptable wetting agents as excipients. Non-limiting examples of surfactants that can be used as wetting agents in compositions of the invention include quaternary ammonium compounds, for example benzalkonium chloride, benzethonium chloride and cetylpyridinium chloride, dioctyl sodium sulfosuccinate, polyoxyethylene alkylphenyl ethers, for example nonoxynol 9, nonoxynol 10, and octoxynol 9, poloxamers (polyoxyethylene and polyoxypropylene block copolymers), polyoxyethylene fatty acid glycerides and oils, for example polyoxyethylene (8) caprylic/capric mono- and diglycerides (e.g., Labrasol™ of Gattefosse), polyoxyethylene (35) castor oil and polyoxyethylene (40) hydrogenated castor oil; polyoxyethylene alkyl ethers, for example polyoxyethylene (20) cetostearyl ether, polyoxyethylene fatty acid esters, for example polyoxyethylene (40) stearate, polyoxyethylene sorbitan esters, for example polysorbate 20 and polysorbate 80 (e.g., Tween™ 80 of ICI), propylene glycol fatty acid esters, for example propylene glycol laurate (e.g., Lauroglycol™ of Gattefossé), sodium lauryl sulfate, fatty acids and salts thereof, for example oleic acid, sodium oleate and triethanolamine oleate, glyceryl fatty acid esters, for example glyceryl monostearate, sorbitan esters, for example sorbitan monolaurate, sorbitan monooleate, sorbitan monopalmitate and sorbitan monostearate, tyloxapol, and mixtures thereof. Such wetting agents, if present, constitute in total about 0.25% to about 15%, preferably about 0.4% to about 10%, and more preferably about 0.5% to about 5%, of the total weight of the composition.

Compositions of the invention optionally comprise one or more pharmaceutically acceptable lubricants (including anti-adherents and/or glidants) as excipients. Suitable lubricants include, either individually or in combination, glyceryl behapate (e.g., Compritol™ 888); stearic acid and salts thereof, including magnesium, calcium and sodium stearates; hydrogenated vegetable oils (e.g., Sterotex™); colloidal silica; talc; waxes; boric acid; sodium benzoate; sodium acetate; sodium fumarate; DL-leucine; PEG (e.g., Carbowax™ 4000 and Carbowax™ 6000); sodium oleate; sodium lauryl sulfate; and magnesium lauryl sulfate. Such lubricants, if present, constitute in total about 0.1% to about 10%, preferably about 0.2% to about 8%, and more preferably about 0.25% to about 5%, of the total weight of the composition.

Magnesium stearate is a preferred lubricant used, for example, to reduce friction between the equipment and granulated mixture during compression of tablet formulations.

Suitable anti-adherents include talc, cornstarch, DL-leucine, sodium lauryl sulfate, colloidal silica, and metallic stearates. Talc is a preferred anti-adherent or glidant used, for example, to reduce formulation sticking to equipment surfaces and also to reduce static in the blend. Talc or colloidal silica, if present, constitute about 0.1% to about 10%, more preferably about 0.25% to about 5%, and still more preferably about 0.5% to about 2%, of the total weight of the composition.

Other excipients such as colorants, flavors and sweeteners are known in the pharmaceutical art and can be used in compositions of the present invention. Tablets can be coated, for example with an enteric coating, or uncoated. Compositions of the invention can further comprise, for example, buffering agents.

Optionally, one or more effervescent agents can be used as disintegrants and/or to enhance organoleptic properties of compositions of the invention. When present in compositions of the invention to promote dosage form disintegration, one or more effervescent agents are preferably present in a total amount of about 2% to about 80%, and more preferably about 4% to about 60%, most preferably about 5% to about 30%, by weight of the composition.

Although unit dose hard capsule and tablet compositions of the invention can be prepared, for example, by direct encapsulation or by direct compression, they may also be granulated prior to encapsulation or compression. Granulation, among other effects, densities milled compositions resulting in improved flow properties, improved compression characteristics and easier metering or weight dispensing of the compositions for encapsulation or tableting. The secondary particle size resulting from granulation (i.e., granule size) is not narrowly critical, it being important only that the average granule size preferably is such as to allow for convenient handling and processing and, for tablets, to permit the formation of a directly compressible mixture that forms pharmaceutically acceptable tablets.

The first and second fractions of clindamycin can be intimately coformulated, for example within individual granules that are subsequently encapsulated or compressed into tablets. Alternatively, the first and second fractions can be spatially separated in a composition of the invention. Illustratively, within a single hard capsule there can be separate granules or beads, for example coated beads, containing the drug in immediate-release form or in controlled-release, slow-release, programmed-release, timed-release, pulse-release, sustained-release or extended-release form. Within a single unit dose tablet there can be separate layers containing the drug in immediate-release form or in controlled-release, slow-release, programmed-release, timed-release, pulse-release, sustained-release or extended-release form. For example, a two-layer clindamycin tablet similar to that described for naproxen in above-cited U.S. Pat. No. 5,609,884 can be prepared. It is contemplated that the compositions of the present invention can contain any one of a number of fractions of clindamycin in any one of a number of release forms described above, provided that at least fractions are designed for pulse release, as described above.

### EXAMPLES

The following example is included herein to illustrate the invention or various aspects of the invention. The example is not intended to be limiting

The following study was done to determine how well clindamycin would be absorbed if released into the colon of a subject, compared to other areas of the digestive tract below the stomach. The study was also done in order to look for any adverse effects resulting from any such releases.

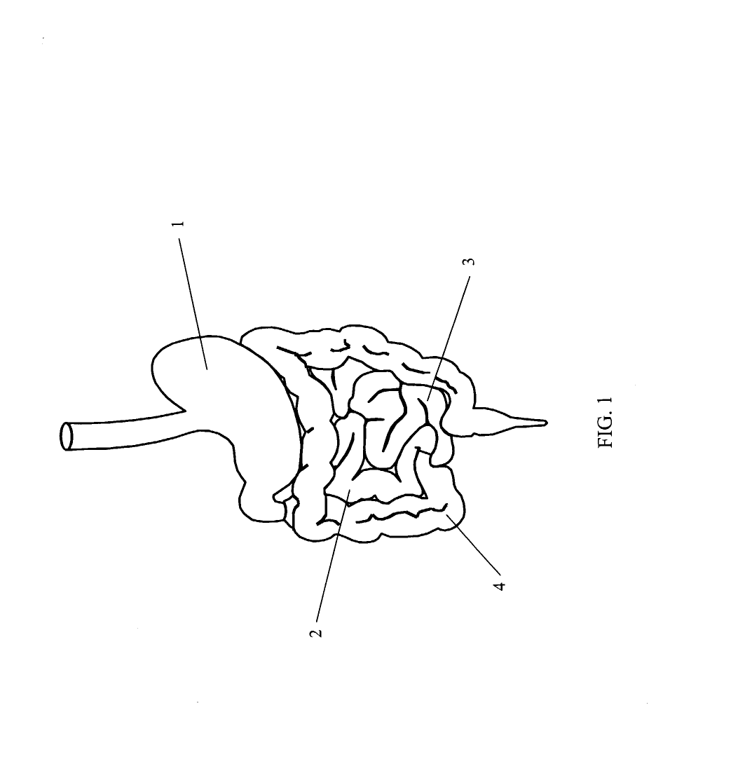

Clindamycin HCl was placed into an Enterion Capsule along with a radioactive tracer. The Enterion Capsule is a mechanical capsule that can pass through the entire digestive tract of a human being without releasing its contents until triggered to do so with a remote control. The capsule, loaded with clindamycin, was orally administered to a subject, tracked until it reached either of two different sites, and triggered to release its contents therein. The sites of a human digestive tract into which clindamycin was released were the stomach and the ascending colon, and these results were compared to the bioavailability of immediate release Cleocin capsules. See FIG. 1 for a diagram illustrating the release sites used in this study, the stomach (1) and the, ascending colon (2).

Results found from the release of clindamycin at the different sites, as described above, are shown in Table 2, below.

TABLE 2

| | Relative Bioavailability |
|---|---|
| | (to immediate release |
| Site of Release | capsules) |
| Stomach | 100% |
| Ascending Colon | 155% |

No adverse side effects of release of clindamycin at any of the sites tested were observed. The level of colonic absorption was considerably higher than expected.

What is claimed is:

1. An extended release composition, comprising a first fraction of clindamycin released in a first pulse, within about thirty minutes of oral administration of the composition to a subject, and a second fraction of clindamycin released in a second pulse, about 4 hours to about 15 hours after oral administration of the composition to the subject.

2. The extended release composition of claim 1, wherein the second fraction of clindamycin is released into the colon of the subject.

3. The extended release composition of claim 1, comprising a tablet in a capsule, wherein the tablet is coated with a functional coating.

4. The extended release composition of claim 3, the tablet further comprising swellable excipients that promote pulsed release of clindamycin by swelling rupture of the functional coating.

5. The extended release composition of claim 3, the tablet further comprising effervescent agents that promote pulsed release of clindamycin by effervescent rupture of the functional coating.

6. The extended release composition of claim 1, wherein the second fraction of clindamycin is present as microparticles, in a form selected from the group consisting of compressed pellets, extruded beads, and spray dried beads.

7. The extended release composition of claim 6, wherein the microparticles are coated.

8. The extended release composition of claim 7, wherein the microparticles further comprise swellable excipients that promote pulsed release of clindamycin by swelling rupture of the microparticle coating.

9. The extended release composition of claim 7, wherein the microparticles further comprise effervescent agents that promote pulsed release of clindamycin by effervescent rupture of the microparticle coating.

10. The extended release composition of claim 7, wherein the microparticle coating is solubilized at a certain pH.

11. The extended release composition of claim 1, wherein the second fraction of clindamycin is contained within beads coated with a release-extending polymer coating.

12. The extended release composition of claim 11, the release-extending polymer coating is selected from the group consisting of: hydroxypropylcellulose, hydroxyethylcellulose, methylhydroxyethylcellulose, methylcellulose, ethylcellulose, cellulose acetate, sodium carboxymethylcellulose, polymers and copolymers of acrylic acid and methacrylic acid and esters thereof.

13. The extended release composition of claim 11, wherein the release-extending polymer coating further comprises a water-soluble substance that forms pores or channels in the coating after oral administration of the composition to a subject.

14. The extended release composition of claim 11, further comprising a suspension of the first fraction of clindamycin and a disintegrant spray coated onto the release-extending polymer coating of the beads.

15. The extended release composition of claim 11, wherein the release-extending polymer coating comprises a water-soluble substance that forms pores or channels in the coating after oral administration of the composition to a subject.

16. The extended release composition of claim 11, further comprising a suspension of the first fraction of clindamycin and a disintegrant spray coated onto the release-extending polymer coating of the beads.

17. The extended release composition of claim 1, wherein the first fraction of clindamycin and the second fraction, of clindamycin are independently is selected from the group consisting of clindamycin HCl, clindamycin phosphate, clindamycin palmitate, and clindamycin crystalline free base.

18. The extended release composition of claim 1, the first fraction of clindamycin and the second fraction of clindamycin both comprising clindamycin HCl.

19. An extended release composition, comprising a first fraction of clindamycin HCl released in a first pulse, within about thirty minutes of oral administration of the composition to a subject, and a second fraction of clindamycin HCl released at least 12 hours after oral administration to the subject.

20. The extended release composition of claim 19, comprising a tablet in a capsule, wherein the tablet is coated with a functional coating.

21. The extended release composition of claim 20, the tablet further comprising swellable excipients that promote pulsed release of clindamycin by swelling rupture of the functional coating.

22. The extended release composition of claim 20, the tablet further comprising effervescent agents that promote pulsed release of clindamycin by effervescent rupture of the functional coating.

23. The extended release composition of claim 19, wherein the second fraction of clindamycin is present as microparticles, in a form selected from the group consisting of compressed pellets, extruded beads, and spray dried beads.

24. The extended release composition of claim 23, wherein the microparticles are coated.

25. The extended release composition of claim 23, wherein the microparticles further comprise swellable excipients that promote pulsed release of clindamycin by swelling rupture of the microparticle coating.

26. The extended release composition of claim 23, wherein the microparticles further comprise effervescent agents that promote pulsed release of clindamycin by effervescent rupture of the microparticle coating.

27. The extended release composition of claim 23, wherein the microparticle coating is solubilized at a certain pH.

28. The extended release composition of claim 19, wherein the second fraction of clindamycin is contained within beads coated with a release-extending polymer coating.

29. The extended release composition of claim 28, the release-extending polymer coating is selected from the group consisting of: hydroxypropylcellulose, hydroxyethylcellulose, methylhydroxyethylcellulose, methylcellulose, ethylcellulose, cellulose acetate, sodium carboxymethylcellulose, polymers and copolymers of acrylic acid and methacrylic acid and esters thereof.

30. The extended release composition of claim 28, wherein the release-extending polymer coating further comprises a water-soluble substance that forms pores or channels in the coating after oral administration of the composition to a subject.

31. The extended release composition of claim 28, further comprising a suspension of the first fraction of clindamycin and a disintegrant spray coated onto the release-extending polymer coating of the beads.

32. The extended release composition of claim 28, wherein the release-extending polymer coating comprises a water-soluble substance that forms pores or channels in the coating after oral administration of the composition to a subject.

33. The extended release composition of claim 28, further comprising a suspension of the first fraction of clindamycin and a disintegrant spray coated onto the release-extending polymer coating of the beads.
